(19) United States
(12) Patent Application Publication
(10) Pub. No.: US 2005/0080303 A1
(43) Pub. Date: Apr. 14, 2005

(54) Porous calcium fluoride, its producing method, catalyst for hydrogenation reaction, and method for producing trihydrofluorocarbon

(76) Inventors: Akira Sekiya, Tsukuba-shi (JP); Masanori Tamura, Tsukuba-shi (JP); Toshiro Yamada, Tokyo (JP)

Correspondence Address: BIRCH STEWART KOLASCH & BIRCH, PO BOX 747, FALLS CHURCH, VA, 22040-0747, US

(21) Appl. No.: 10/680,257

(22) Filed: Oct. 8, 2003

(51) Int. Cl. C07C 17/10

(52) U.S. Cl. 570/176; 423/497

(57) ABSTRACT

Porous calcium fluoride having a large surface area, a method for producing the same, a catalyst (for hydrogenation reaction in particular) using the porous calcium fluoride as a carrier with superior activity, selectivity, and durability, and a method for producing trihydrofluorocarbon using the catalyst. The porous calcium fluoride having a BET surface area of 20 m²/g to 200 m²/g is prepared by reacting soda lime with hydrogen fluoride. The carried cataryst (for hydrogenation reaction in particurar) is obtained by causing a metal or metal compound to be carried on carrier formed of the porous calcium fluoride. Trihydrofluorocarbon (2) is produced by causing a fluooroalkene (1) to contact hydrogen in the presence of the catalyst for hydrogenation reaction. 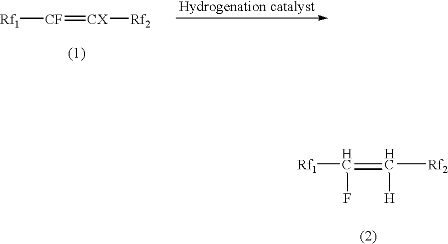
wherein X denotes a halogen atom, Rf₁ and Rf₂ individually denote a fluorine or a parafluoroalkyl group, and Rf₁ may be bonded to Rf₂ to form a ring.

### BACKGROUND OF THE INVENTION

1. Field of the Invention

The present invention relates to porous calcium fluoride, a method for producing the same, a carried catalyst using the calcium fluoride as a carrier such as a hydrogenation catalyst, and a method for producing trihydrofluorocarbon using the hydrogenation catalyst.

2. Description of Background Art

Calcium fluoride (CaF₂) has been conventionally known to be useful as a carrier for a carried catalyst (a catalyst obtained by causing a metal or metal compound to be carried on a carrier) since it is inert to various reactions using hydrogen gas or oxygen gas.

For example, in Published Japanese Translation of PCT International Publication for Patent Application No. 2001-500051, an alkaline earth metal-containing compound such as calcium fluoride or calcium molybdate is used as a carrier for a silver catalyst for oxidizing propylene into propylene oxide in a vapor phase.

Japanese Patent Application Laid-open No. 6-145114 describes an alcohol reacted with carbon monoxide in the presence of a solid catalyst using a metal halide such as calcium fluoride as a carrier to obtain a carbonic acid diester.

Japanese Patent Application Laid-open No. 7-69943 describes a method for producing 1,1,3,3,3-pentafluoropropane comprising reacting 1,1,3,3,3-pentafluoropropene with hydrogen in the presence of a metal oxide catalyst using a vapor phase method in the temperature range of 40° C. to 300° C. As a carrier to carry a metal oxide thereon, calcium fluoride, alumina, aluminum fluoride, or the like is used.

Japanese Patent Application Laid-open No. 7-112944 describes a method for producing hexafluorocyclobutene comprising dechlorinating 1,2-dichlorohexafluorocyclobutane with hydrogen in the presence of a metal oxide catalyst and/or a silicon oxide catalyst. As a carrier for the metal oxide catalyst used in this reaction, calcium fluoride, activated carbon, alumina, or the like is used.

Calcium fluoride is found as a fluorite (pure form) or a fluorspar (ore) in the natural environment. However, calcium fluoride can be artificially produced. As a conventional method for producing calcium fluoride, a method comprising reacting a soluble calcium salt with sodium fluoride, a method comprising dissolving calcium carbonate or calcium hydroxide in hydrofluoric acid and evaporating the solution, and the like are known.

To cause a carried substance (metal or metal compound) in an increased amount to be carried on a carrier to improve catalytic activity of a carried catalyst, the carrier must have a larger surface area. However, since there is a certain limitation to the surface area of conventional calcium fluoride, naturally occurring or artificially produced as mentioned above, there is a certain limitation to the amount of the metal or metal compound carried thereon.

The present invention has been achieved in view of such a situation. An object of the present invention is to provide porous calcium fluoride having a surface area of 20 m²/g to 200 m²/g, a method for producing porous calcium fluoride, a carried catalyst (hydrogenation catalyst in particular) using the porous calcium fluoride as a carrier, and a method for producing trihydrofluorocarbon using the hydrogenation catalyst.

### SUMMARY OF THE INVENTION

The present inventors have found that (i) porous calcium fluoride can be obtained by causing soda lime to contact hydrogen fluoride at a specific temperature and the porous calcium fluoride obtained in this manner has a surface area remarkably larger than that of conventional calcium fluoride, (ii) a hydrogenation catalyst excelling in catalytic activity and durability can be obtained by causing a noble metal or noble metal compound to be carried on the obtained porous calcium fluoride, and (iii) a fluoroalkene can be hydrogenated with high selectivity and at a high yield using the obtained hydrogenation catalyst. These findings have led to the completion of the present invention.

Specifically, the present invention provides:
(1) porous calcium fluoride having a surface area measured using a BET method of 20 m²/g to 200 m²/g;
(2) a method for producing porous calcium fluoride comprising causing soda lime to contact hydrogen fluoride;
(3) a carried catalyst obtained by causing a metal or metal compound to be carried on a carrier formed of the porous calcium fluoride;
(4) a hydrogenation catalyst obtained by causing a noble metal or noble metal compound to be carried on a carrier formed of the porous calcium fluoride; and
(5) a method for producing trihydrofluorocarbon comprising causing a fluoroalkene of the formula (1):
   Rf₁—CF═CX—Rf₂ (1)
   wherein X represents a halogen atom, Rf₁ and Rf₂ individually represent a fluorine atom or perfluoroalkyl group, and Rf₁ may be bonded to Rf₂ to form a ring, to contact hydrogen in the presence of the hydrogenation catalyst to produce trihydrofluorocarbon of the formula (2):
   Rf₁—CHF—CH₂—Rf₂ (2)
   wherein Rf₁ and Rf₂ are as defined above.

### DETAILED DESCRIPTION OF THE INVENTION

The inventions (1)-(5) will be described in detail below.

(1) Porous Calcium Fluoride

Calcium fluoride used in the present invention is porous. Generally, the term “porous” refers to a state in which many small voids in the form of bubbles are present in a solid or on the surface of a solid. The surface area of the porous calcium fluoride of the present invention measured using a BET method is 20 m²/g to 200 m²/g, preferably 30 m²/g to 150 m²/g, more preferably 40 m²/g to 100 m²/g, and still more preferably 60 m²/g to 80 m²/g. If the surface area measured using the BET method is less than 20 m²/g, the amount of a metal or metal compound catalyst carried is insufficient and the catalyst may exhibit insufficient catalytic activity. If the surface area exceeds 200 m²/g, the catalyst exhibits increased catalytic activity but may exhibit decreased catalytic strength.

The surface area of calcium fluoride can be measured using a BET method by gas adsorption. Specifically, provided that various gases such as nitrogen gas, oxygen gas, and argon gas are adsorbed in calcium fluoride at near their boiling points to obtain an adsorption isotherm curve of which the point starting the straight line is a B point and the amount of gases adsorbed at the B point is νₘ (standard conditions), the surface area S can be determined using the formula:
S=0.41 νₘ(M/D)^{2/3}
wherein M represents the molecular weight of the adsorbed molecule and D represents the density of the adsorbate at an adsorption temperature.

The surface area S can be determined, if the B point is unclear, using a BET equation (Brunauer-Emmett-Teller equation) by plotting measured values p/p₀ along the horizontal axis and p/v (p₀−p)×10³ along the vertical axis, determining νₘ from the slope (c−1)/νₘc and the gradient 1/νₘc of the obtained straight line, and applying the determined νₘ to the following formula:
S=(νₘ/22,400)×N×a
wherein N represents Avogadro's number and a represents the surface area occupied by one adsorbed molecule. Here, p represents adsorption equilibrium pressure, p₀ represents saturated vapor pressure of the adsorbed molecule at a measurement temperature, c represents a constant related to adsorption heat, and ν represents the amount of adsorption (reduced to standard conditions) at the pressure p.

The pore volume Vs of the porous calcium fluoride of the present invention is in the range of usually 0.05 cm³/g to 0.5 cm³/g, preferably 0.06 cm³/g to 0.4 cm³/g, and more preferably 0.08 cm³/g to 0.2 cm³/g. Examples of the method for determining the pore volume Vs include (i) a method comprising placing calcium fluoride with a total volume V1 in a space filled with helium with specific volume and pressure, determining the real volume V2 from the increase in the pressure, and calculating the pore volume Vs from the formula, Vs=V1−V2, (ii) a method comprising placing calcium fluoride in a space filled with helium with a specific volume, measuring the volume of the space filled with helium, measuring the volume of the space filled with mercury after discharging the helium, and calculating the pore volume Vs from the difference between the two measured volumes, and (iii) a method comprising causing a saturated vapor or liquid to be absorbed in calcium fluoride with a specific weight, removing the solution adhering to the surface, and calculating the pore volume Vs from the increase in weight by the removal. Of these, the method (ii) is generally used.

The average pore diameter of the porous calcium fluoride of the present invention is in the range of usually 20-200 Å, preferably 30-180 Å, and more preferably 50-150 Å. The average pore diameter can be determined from the formula 4×Vs/S. Here, Vs represents a pore volume and S represents a surface area. The average pore diameter can be determined using a pore diameter automatic measuring instrument (for example, NOVA 1000, manufactured by Yuasa-Ionics Co., Ltd.).

The porous calcium fluoride of the present invention can be produced by causing soda lime to contact hydrogen fluoride at a specific temperature. Soda lime is a white particulate solid exhibiting strong basicity obtained by immersing quicklime in a concentrated solution of sodium hydroxide and heating the mixture. The soda lime is used as an absorbent for carbon dioxide or water and is commercially available. There are no specific limitations to the form of the soda lime used in the present invention. Examples of the form include a powder and a pellet. Of these, a pellet is preferable.

There are no specific limitations to the method for causing soda lime to contact hydrogen fluoride at a specific temperature. Examples of the method include (a) a method comprising causing solid soda lime such as a soda lime powder or pellet to contact hydrogen fluoride gas at a specific temperature and (b) a method comprising adding a specific amount of hydrofluoric acid to a soda lime solution and removing the solvent by evaporation. Of these, the method (a) is preferable in the present invention, since anhydrous porous calcium fluoride having a specific surface area can be efficiently obtained.

The temperature at which soda lime is caused to contact hydrogen fluoride is in the range of usually 0° C. to 400° C., preferably 20° C. to 400° C., and more preferably 30° C. to 280° C. There are no specific limitations to the amount of hydrogen fluoride used inasmuch as the amount is sufficient for reacting with soda lime to produce calcium fluoride.

In the method for producing porous calcium fluoride in the present invention, it is preferable that soda lime be heated in a nitrogen gas atmosphere at 150° C. to 250° C. for 1-10 hours, dried sufficiently, and caused to contact hydrogen fluoride gas. In this case, anhydrous hydrogen fluoride is preferably used. Hydrogen fluoride diluted with a diluent gas such as nitrogen gas or argon gas may be used. Although there are no specific limitations, the dilution rate of hydrogen fluoride is at a volume ratio of inert gas to hydrogen fluoride in the range of usually 10:1 to 1:10, and preferably 5:1 to 1:5.

According to this production method, porous calcium fluoride having a surface area measured using a BET method of 20 m²/g to 200 m²/g can be obtained.

(2) Carried Catalyst

The porous calcium fluoride of the present invention is useful as a carrier for various metals or metal compounds. Examples of the metal that can be carried include chromium, iron, cobalt, copper, nickel, manganese, palladium, rhodium, ruthenium, rhenium, platinum, iridium, and osmium. Examples of the metal compound include an acetate, sulfate, nitrate, halide, oxide, and hydroxide of these metals.

The carried catalyst of the present invention exhibits activity to various catalytic reactions based on these metals or metal compounds higher that that of a conventional carried catalyst. A carried catalyst carrying a noble metal or noble metal compound is particularly useful as a hydrogenation catalyst. Examples of the carried noble metal or noble metal compound used as a hydrogenation catalyst include noble metals such as palladium, rhodium, ruthenium, rhenium, platinum, iridium, and osmium and noble metal compounds such as palladium acetate, palladium sulfate, palladium nitrate, palladium chloride, and platinum oxide.

Of these, palladium and palladium compounds are particularly preferable.

As these catalysts, a catalyst formed of a single metal, bimetal catalyst, alloy catalyst, or the like may be used. The carried catalyst of the present invention used as a hydrogenation catalyst preferably contains palladium as a main component.

The above hydrogenation catalyst may further contain a metal component other than the above noble metal (an additional metal component). Examples of the additional metal component include silver, copper, gold, tellurium, zinc, chromium, molybdenum, thallium, tin, bismuth, and lead. The amount of the additional metal component is 0.01-500 parts by weight, and preferably 0.1-300 parts by weight, for 100 parts by weight of the above metal. This can allow metallic properties to be sufficiently exhibited. Generally, when two or more metals or metal compounds are used, properties of the component elements can be exhibited or catalytic activity can be varied according to the composition.

When a metal or metal compound is caused to be carried on porous calcium fluoride, the form of porous calcium fluoride as a carrier may be a powder, a sphere, or a particulate such as a pellet. The particulate may be either a formed product or a crushed product. The amount of the metal or metal compound carried is in the range of usually 0.05-20 wt %, preferably 0.1-10 wt %, and more preferably 0.5-7 wt % for the carrier.

Generally, catalytic activity can increase according to an increase in the amount of a metal or metal compound carried in a catalyst. Porous calcium fluoride obtained in the present invention, having a surface area measured using a BET method of 20 m²/g to 200 m²/g, can remarkably increase the amount of a metal or metal compound carried.

As a method for causing a metal or metal compound to be carried on the porous calcium fluoride of the present invention, a conventional method such as an ion exchange method or an impregnation method can be used, for example. For example, a method comprising mixing a porous calcium fluoride carrier with an aqueous solution of a metal compound at a specific ratio, drying the mixture, and processing the dried product at a high temperature of 100° C. to 600° C. to cause the metal compound to be carried on the carrier can be given. When a metal compound is to be carried, an individual aqueous solution of a metal compound or a mixture of an aqueous solution of a metal compound and an optional aqueous solution of an additional metal at a desired ratio and a desired concentration may be used.

After the metal compound is caused to be carried on porous calcium fluoride, the carried catalyst can be activated by reducing the catalyst using a wet reduction method or a vapor phase reduction method. In the wet reduction method, after the metal compound as a catalytic component is caused to be carried on the carrier, a suitable reducing agent is added to the carried catalyst to cause reduction at room temperature. Examples of the reducing agent used include formalin, hydrazine, formic acid, and sodium borohydride. In the vapor phase reduction method, after the metal compound is caused to be carried on the carrier, the carried catalyst is processed in a hydrogen stream at 100° C. to 600° C. to cause reduction.

(3) Preparation of Trihydrofluorocarbon

The hydrogenation catalyst of the present invention can be suitably used for preparing trihydrofluorocarbon of the formula (2), Rf₁—CHF—CH₂—Rf₂, by causing a fluoroalkene of the formula (1), Rf₁—CF═CX—Rf₂, to contact hydrogen.

In the above formula (1), X represents a halogen atom such as a fluorine atom, chlorine atom, bromine atom, or iodine atom. In the present invention, X is preferably a fluorine atom or chlorine atom.

Rf₁ and Rf₂ individually represent a fluorine atom or a fluoroalkyl group obtained by substituting some or all hydrogen atoms in an alkyl group with fluorine atoms. As such a fluoroalkyl group, a fluoroalkyl group having 1-20 carbon atoms is preferable. Examples of the preferable fluoroalkyl group include a trifluoromethyl group, 1,1,1-trifluoroethyl group, pentafluoroethyl group, heptatluoro-n-propyl group, heptafluoroisopropyl group, nonafluoro-n-butyl group, nonafluoro-sec-butyl group, nonafluoro-t-butyl group, undecafluoro-n-pentyl group, undecafluoroneopentyl group, perfluorohexyl group, perfluoroheptyl group, perfluorooctyl group, perfluorononyl group, and perfluorodecyl group.

The fluoroalkene of the formula (1) may form a ring having 4-8 carbon atoms by bonding Rf₁ to Rf₂. Compounds forming such a ring include a cyclobutene compound, cyclopentene compound, cyclohexene compound, cycloheptene compound, and cyclooctene compound.

Specific examples of the compound of the formula (1) include linear fluoroalkenes such as 1-chloro-1,2,3,3,4,4,4-heptafluorobutene-1, 2-chloro-1,1,3,3,4,4,4-heptafluorobutene-1, 2-chloro-1,1,1,3,4,4,4-heptafluorobutene-2, 1-chloro-1,2,3,3,4,4,5,5,5-nonafluoropentene-1, 2-chloro-1,1,3,3,4,4,5,5,5-nonafluoropentene-1, 2-chloro-1,1,1,3,4,4,5,5,5-nonafluoropentene-2, 3-chloro-1,1,1,2,4,4,5,5,5-nonafluoropentene-2, 1,1,2,3,3,4,4,5,5,5-decafluoropentene-1, 1,1,1,2,3,4,4,5,5,5-decafluoropentene-2, 1-chloro-1,2,3,3,4,4,5,5,6,6,6-undecafluorohexene-1, 2-chloro-1,1,3,3,4,4,5,5,6,6,6-undecafluorohexene-1, 2-chloro-1,1,1,3,4,4,5,5,6,6,6-undecafluorohexene-2, 3-chloro-1,1,1,2,4,4,5,5,6,6,6-undecafluorohexene-2, and 3-chloro-1,1,1,2,2,4,5,5,6,6,6-undecafluorohexene-3; and cyclic fluoroalkenes such as 1-chloro-2,3,3,4,4-pentafluorocyclopentene-1, 1-chloro-2,3,3,4,4,5,5-heptafluorocyclopentene-1, 1,2,3,3,4,4-hexafluorocyclopentene-1, 1,2,3,3,4,4,5,5-octafluorocyclopentene-1, and 1-chloro-2,3,3,4,4,5,5,6,6-nonafluorocyclohexene-1.

Hydrogen is advantageously used in the hydrogenation in an amount in excess of the compound of the above formula (1). For example, the amount of hydrogen used is two mols or more, and preferably 2-50 mols, for one mol of the compound of the above formula (1).

As a method for hydrogenation, liquid phase reaction or vapor phase reaction can be used. In the liquid phase reaction, a solvent may be used. In the vapor phase reaction, a diluent may be optionally used. A method such as fixed bed vapor phase reaction or fluid bed vapor phase reaction may be applied to the vapor phase reaction.

There are no specific limitations to the solvent used in the liquid phase reaction. Examples of the solvent used include an aliphatic hydrocarbon, aromatic hydrocarbon, hydrofluorocarbon, alcohol, ether, ketone, ester, and water.

Examples of the aliphatic hydrocarbon include linear or cyclic hydrocarbons having 4-15 carbon atoms such as n-butane, n-pentane, methylpentane, n-hexane, cyclopentane, and cyclohexane. Examples of the aromatic hydrocarbon include trifluoromethylbenzene. Examples of the hydrofluorocarbon include pentafluoroethane, pentafluoropropane, hexafluorobutane, and decafluoropentane. Examples of the alcohol include methanol, ethanol, n-propanol, isopropanol, n-butanol, sec-butanol, t-butanol, and cyclopentanol. Examples of the ether include diethyl ether, diisopropyl ether, and ethylene glycol dimethyl ether. Examples of the ketone include acetone, methyl ethyl ketone, methyl isobutyl ketone, and cyclopentanone. Examples of the ester include methyl acetate, ethyl acetate, propyl acetate, and methyl propionate. These solvents may be used either individually or in combination of two or more. Although there are no specific limitations to the amount of the solvent used, the solvent is usually used in an amount of 0-80 parts by weight, and preferably 0-50 parts by weight, for 100 parts by weight of the fluoroalkene of the formula (1).

In the vapor phase reaction, hydrogen gas can be either used alone or diluted with a diluent gas. Any diluent gas inert to the hydrogenation can be used. Examples of the diluent used include nitrogen gas, rare gas, hydrocarbon gas, and hydrofluorocarbon gas. These diluents may be used either individually or in combination of two or more. Although there are no specific limitations to the amount of the diluent used, the amount used is usually 0-500 parts by weight, and preferably 0-200 parts by weight, for 100 parts by weight of the compound of the above formula (1).

The pressure in the reaction system of the hydrogenation is generally in the range of usually atmospheric pressure to 50 kgf/cm², and preferably atmospheric pressure to 20 kgf/cm². The reaction temperature is in the range of usually room temperature to 350° C., and more preferably room temperature to 200° C. In this hydrogenation, the reaction system is preferably stirred or shaken, if required.

Either a batch reaction or a continuous reaction in which a raw material is continuously supplied to a reaction vessel (reaction tube) and the reaction product is continuously discharged from the reaction vessel can be preferably applied to the hydrogenation of the present invention. The reaction vessel used in the batch reaction is a pressure vessel. In the continuous reaction, one or more reaction vessels such as cascade reaction vessels connected in series can be used. Examples of the suitable material for the reaction vessel include stainless steel and inconel. These reaction vessels are preferably conditioned by nitric acid treatment, for example, before use.

In this reaction, an acidic component such as hydrogen chloride is produced as a by-product. This acidic component is preferably removed by absorption or neutralization during the reaction. To remove the acidic component, an additive can be added to the reaction system. Examples of the additive used include a hydroxide, oxide, weak acid salt, and organic acid salt of an alkaline metal or alkaline earth metal, more specifically, soda lime, calcined lime, alkali carbonate, and alkali acetate.

After terminating the reaction, the acidic component is optionally absorbed or neutralized using an additive and the target product can be optionally using a conventional purification method such as distillation.

The compound of the above formula (2) obtained using the production method of the present invention is a linear or alicyclic compound having four or more carbon atoms containing a —CH₂—CHF— group in the molecule. The method of the present invention is particularly suitably applied to the synthesis of an alicyclic compound. The number of carbon atoms in the basic skeleton of the compound containing a —CH₂—CHF— group is usually 4-10, preferably 4-6, and particularly preferably 5.

Specific examples of the linear compound containing a —CH₂—CHF— group include 1, 1,1,2,5,5,5-heptafluoro-n-pentane, 1,1,1,2,2,3,5,5,5-nonafluoro-n-pentane, 1,1,1,2,2,4,5,5,5-nonafluoro-n-pentane, 1,1,1,2,2,3,3,4,6,6,6-undecafluoro-n-hexane, and 1,1,1,2,2,3,3,5,6,6,6-undecafluoro-n-hexane.

As the alicylic compound of the above formula (2), a compound of the formula (3) can be given.

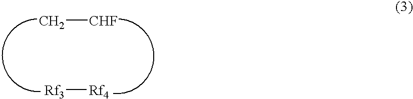

In the formula (3), Rf₃ and Rf₄ individually represent a fluoroalkylene group having 1-3 carbon atoms. Examples of the fluoroalkylene group having 1-3 carbon atoms include a fluoromethylene group, difluoromethylene group, fluoroethylene group, difluoroethylene group, trifluoroethylene group, tetrafluoroethylene group, fluorotrimethylene group, difluorotrimethylene group, trifluorotrimethylene group, tetrafluorotrimethylene group, pentafluorotrimethylene group, and hexafluorotrimethylene group.

Specific examples of the compound of the above formula (3) include alicyclic compounds such as 1,1,2,2,3-pentafluorocyclobutane, 1,1,2,2,3,3,4-heptafluorocyclopentane, and 1,1,2,2,3,3,4,4,5-nonafluorocyclohexane. Of these compounds, 1,1,1,2,2,3,5,5,5-nonafluoro-n-pentane and 1,1,2,2,3,3,4-heptafluorocyclopentane are preferable, and 1,1,2,2,3,3,4-heptafluorocyclopentane are particularly preferable.

### EXAMPLES

The present invention is described in more detail by way of examples. However, the following examples should not be construed as limiting the present invention.

### Examples 1-4

### Preparation of Porous Calcium Fluoride

A suitable amount of commercially available soda lime in the form of a pellet was placed in a reaction tube made of inconel (internal diameter: 4 mm, length: 200 mm). Dry nitrogen gas was circulated in the reaction tube at 200° C. for three hours to dry the soda lime. Then, the gas in the reaction tube was replaced with a gas mixture of nitrogen gas and anhydrous hydrogen fluoride (volume ratio: 3:1). The gas mixture was circulated at 40° C. for three hours. Next, the gas in the reaction tube was replaced with anhydrous hydrogen fluoride. The reaction temperature was raised to 250° C. over two hours and the gas was heated at the same temperature for 10 hours. After terminating the reaction, dry nitrogen gas was circulated in the reaction tube for 10 hours and the gas in the reaction tube was fully replaced with nitrogen gas (nitrogen washing). As a result, the target porous calcium fluoride was obtained. The surface area (m²/g) measured by a BET method, pore volume Vs (cm³/g), and average pore diameter (A) of the obtained porous calcium fluoride and the sources of the commercially available soda limes used as raw materials are collectively shown in Table 1. In Table 1, a, b, c, and d represent soda limes respectively manufactured by Kanto Kagaku Co., Ltd., Merck & Co., Inc., Wako Pure Chemical Industries, Ltd., and Nakarai Tesque, Inc. And Vs represents a pore volume.

TABLE 1

| | BET surface area | Vs | Average pore | |
|---|---|---|---|---|
| | (m²/g) | (cm³/g) | diameter (Å) | Soda lime |
| Example 1 | 60.252 | 0.1451 | 96.30 | a |
| Example 2 | 28.042 | 0.0881 | 125.68 | b |
| Example 3 | 61.634 | 0.1489 | 96.62 | c |
| Example 4 | 91.446 | 0.1457 | 63.75 | d |

### Example 5

### Preparation of Hydrogenation Catalyst A

10 g of porous calcium fluoride obtained in Example 1 was weighed in a 100 ml-eggplant flask. 10 ml of a 35% aqueous solution of hydrochloric acid and distilled water were added to the flask so that the porous calcium fluoride was entirely under the surface of the liquid mixture. The mixture was then allowed to stand for eight hours. Next, the solution was removed by suction filtration. The filtrate was washed with 20 ml of distilled water three times.

500 mg of palladium chloride (II) was dissolved in 3 ml of a 35% aqueous solution of hydrochloric acid. This solution was added to the eggplant flask containing the porous calcium fluoride treated with the aqueous solution of hydrochloric acid and these components were sufficiently mixed. The mixture was allowed to stand for 12 hours. As a result, the palladium chloride was impregnated in the porous calcium fluoride.

The obtained mixture (porous calcium fluoride impregnated with palladium chloride) was heated to 60° C. under reduced pressure (20 mmHg) and dried. Then, the dried product was placed in a reaction tube made of inconel (internal diameter: 4 mm, length: 200 mm) and the gas in the tube was replaced with nitrogen at 150° C. for three hours (nitrogen flow rate: 40 ml/minute). Next, the replacement gas was changed from nitrogen gas to hydrogen gas. Hydrogen gas was circulated in the tube at 150° C. for five hours (hydrogen flow rate: 40 ml/minute). In this manner, a hydrogenation catalyst A of Example 5 was prepared. The amount of palladium carried in this hydrogenation catalyst was 3 wt %.

### Example 6

### Preparation of Heptafluorocyclopentane

0.41 g of the hydrogenation catalyst A prepared in Example 5 was placed in a reaction tube made of inconel (internal diameter: 4 mm, length: 200 mm). The catalyst was activated by heating at 150° C. for two minutes in a nitrogen atmosphere and then in a hydrogen atmosphere for 10 minutes. Next, 1-chloro-2,3,3,4,4,5,5-heptafluorocyclopentene-1 (hereinafter abbreviated as “MCL”) and hydrogen gas were added from one inlet port of the reaction tube respectively at flow rates of 0.21 g/minute and 200 ml/minute at a reaction tube temperature of 150° C. (The contact time of the gases with the catalyst was 0.1 second.) After a specific period of time had elapsed since the start of the reaction, the gas discharged from one outlet port of the reaction tube was collected in a liquid nitrogen trap. The collected gas was analyzed using gas chromatography (hereinafter abbreviated as “GC”). The results of analysis are shown in Table 2 together with the reaction temperature and the reaction time.

### Comparative Example 1

### Preparation of Heptafluorocyclopentane

MCL was hydrogenated in the same manner as in Example 6, except that 0.25 g of commercially available palladium carried on activated hydrocarbon (the amount of palladium carried: 5 wt %, catalyst B) was used instead of the hydrogenation catalyst obtained in Example 5. The GC analysis results of the reaction product, the reaction temperature, and the reaction time are shown in Table 2. The following abbreviations can be applied to Table 2.

F7A: 1,1,2,2,3,3,4-heptafluorocyclopentane

F7E: 1,3,3,4,4,5,5-heptafluorocyclopentene

F6A: 1,1,2,2,3,3-hexa fluorocyclopcntane

TABLE 2

| Hydro- | Reaction | | | | | |
|---|---|---|---|---|---|---|
| genation | tempera- | Reaction time | Reaction product (%) | | | |
| catalyst | ture (° C.) | (minutes) | F7A | F7E | F6A | MCL |
| Catalyst A | 150 | 10 | 96.81 | 0.67 | 2.00 | 0.17 |
| | | 40 | 96.31 | 1.78 | 1.23 | 0.45 |
| | | 70 | 95.42 | 2.57 | 1.12 | 0.63 |
| | | 100 | 95.71 | 2.16 | 1.30 | 0.5 |
| | | 150 | 91.79 | 5.63 | 0.67 | 1.38 |
| Catalyst B | 150 | 10 | 88.35 | 10.27 | 0.37 | 0.87 |
| | | 40 | 86.73 | 9.97 | 0.30 | 0.85 |
| | | 70 | 87.8 | 10.80 | 0.23 | 1.02 |
| | | 100 | 87.8 | 10.77 | 0.18 | 1.09 |
| | | 150 | 82.05 | 14.44 | 0.16 | 2.50 |

As is clear from Table 2, the target 1,1,2,2,3,3,4-heptafluorocyclopentane can be obtained in a shorter period of time with higher selectivity by using the hydrogenation catalyst carried on the porous calcium fluoride of the present invention (catalyst A) rather than the hydrogenation catalyst of Comparative Example (catalyst B). The hydrogenation catalyst A of the Examples has a catalyst life longer than that of the hydrogenation catalyst B of the Comparative Example even at a high temperature of 150° C.

As described above, the present invention provides porous calcium fluoride having a BET surface area of 20 m²/g to 200 m²/g, a method for producing porous calcium fluoride, and a carried catalyst using porous calcium fluoride as a carrier.

When the carried catalyst of the present invention is a hydrogenation catalyst, the catalyst of the present invention can produce trihydrofluorocarbon with excellent durability, at a high yield, and with high selectivity, as compared with a conventional hydrogenation catalyst.

1. Porous calcium fluoride having a surface area measured using a BET method of 20 m²/g to 200 m²/g.

2. A method for producing porous calcium fluoride comprising causing soda lime to contact hydrogen fluoride.

3. A carried catalyst obtained by causing a metal or metal compound to be carried on a carrier formed of the porous calcium fluoride according to claim 1.

4. A hydrogenation catalyst obtained by causing a noble metal or noble metal compound to be carried on a carrier formed of the porous calcium fluoride according to claim 1.

5. A method for producing trihydrofluorocarbon comprising causing a fluoroalkene of the formula (1):
Rf₁—CF═CX—Rf₂ (1)
wherein X represents a halogen atom, Rf₁ and Rf₂ individually represent a fluorine atom or fluoroalkyl group, and Rf₁ may be bonded to Rf₂ to form a ring, to contact hydrogen in the presence of the catalyst according to claim 4 to produce trihydrofluorocarbon of the formula (2):
Rf₁—CHF—CH₂—Rf₂ (2)
wherein Rf₁ and Rf₂ are as defined above.
